(19) United States
(12) Patent Application Publication
(10) Pub. No.: US 2019/0246964 A1
(43) Pub. Date: Aug. 15, 2019

(54) Combined Non Invasive Blood Glucose Monitor Device

(71) Applicants: Michael Zhang, Winnipeg (CA); Zhenyu Wu, Winnipeg (CA)

(72) Inventors: Michael Zhang, Winnipeg (CA); Zhenyu Wu, Winnipeg (CA)

(21) Appl. No.: 16/279,633

(22) Filed: Feb. 19, 2019

Related U.S. Application Data
(63) Continuation-in-part of application No. 14/509,422, filed on Oct. 8, 2014, now abandoned.
(60) Provisional application No. 61/889,066, filed on Oct. 10, 2013.

(51) Int. Cl. A61B 5/145; A61B 5/1455; A61B 5/01; A61B 5/0205; A61B 5/0285; A61B 5/00

(52) U.S. Cl. CPC A61B 5/14532; A61B 5/14551; A61B 5/7278; A61B 5/02055; A61B 5/0285; A61B 5/01

(57) ABSTRACT

This patent is a combined non-invasive method of monitoring glucose level. Monitoring the concentration of glucose level in human blood and tissue was developed by a combined non-invasive technique. This combined method includes skin temperature and pulse wave measurement. All these measurements are used to calculate the blood glucose level. This method also can be used to measure the diabetes related damage of micro arterial.

### PRIOR APPLICATION INFORMATION

The instant application is a continuation-in-part application of U.S. patent application Ser. No. 14/509,422, filed Oct. 7, 2014 and entitled “A COMBINED NON INVASIVE BLOOD GLUCOSE MONITOR DEVICE”, the contents of which are incorporated herein by reference, which claimed the benefit of U.S. Provisional Patent Application 61/889,066, filed Oct. 10, 2013, now abandoned.

### BACKGROUND OF THE INVENTION

Diabetes is a chronic disease, the treatment of which requires significant healthcare resources. Based on the report of The International Diabetes Federation (IDF), diabetes affected 194 million people in 2003 and is projected to increase to 333 million people in 2025. As diabetes progresses, those inflicted with the disease develop other complications, such as cardiovascular, renal, and eye conditions which require more frequent hospitalizations and increased monitoring of the patients. Because the diabetes epidemic is growing so fast, the public health system is focusing on preventing the continued growth of the high risk population. Maintaining the proper glycaemia metabolic control is the main goal of existing diabetes management models. According to clinical guideline of the WHO, blood glucose level should be measured at least 4 times per day for a normal diabetes patient.

Currently, measuring arterial blood glucose level requires the taking of blood from the finger. It is very uncomfortable and may cause infections. A non-invasive blood glucose level testing device is desired.

### SUMMARY OF THE INVENTION

According to one aspect of the invention, there is provided a non-invasive method of determining blood glucose level in a patient comprising: calculating dissipated heat by measuring room temperature and body temperature of the patient; calculating blood oxygen saturation; determining a first glucose value using the calculated dissipated heat and the blood oxygen saturation; calculating a glucose coefficient using pulse wave analysis; and determining blood glucose level using the first glucose value and the glucose coefficient.

In another aspect of the invention, there is provided a method of determining blood glucose level of an individual comprising: determining body temperature of the individual; determining blood oxygen saturation; calculating blood flow for the individual using a pulse waveform from the individual; calculating a body heat coefficient and a blood flow coefficient by comparing the waveform from the individual to at least one waveform from a library of waveforms; and calculating the blood glucose level of the patient from the body temperature, blood flow, oxygen saturation, body heat coefficient and blood flow coefficient.

### BRIEF DESCRIPTION OF THE DRAWINGS

FIG. 1 Distance of two waveforms. (a) and (d) in FIG. 1 are two waveforms to compare. (b) point to point differenced. (c) shows the point based warping result based on dynamic time warping.

FIG. 2. Matrix of point to point distance between two pulse waveforms.

FIG. 3 illustrates the pulse wave signal detected at the finger.

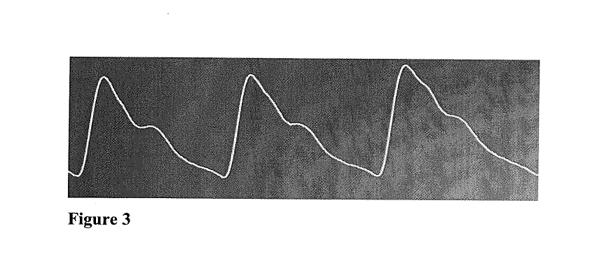

FIG. 4 is a flow chart diagram of the device.

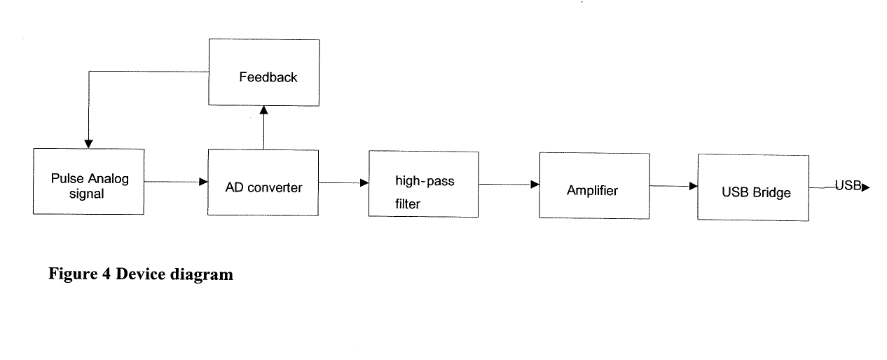

FIG. 5 is a flow chart diagram of the AD converter.

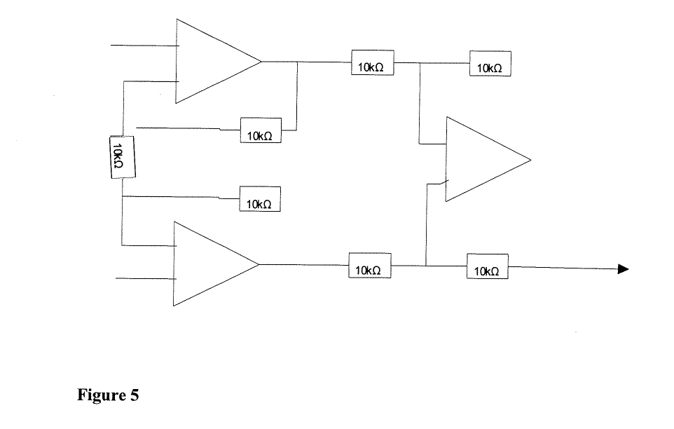

FIG. 6 is an example showing measurements from one patient.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

As discussed herein, the blood glucose level of a patient is measured by using a pulse wave sensor to detect the blood flow at the index finger and then tracking the strength of the flow using pulse wave data. As discussed herein, to record the pulse wave, patients were comfortably seated with the right hand supported. A pulse wave sensor was applied to the index finger of the right hand. Only the appropriate and stable contour of the pulse wave was recorded, as discussed below. As will be known to one of skill in the art, there are many different accepted definitions of a stable waveform or stable contour. For example, in some embodiments, a contour may be considered stable when at least 10 consecutive contours vary in height, shape and/or size by less than 5%. The recorded pulse wave shape was analysed to extract the size of each pulse, the distance between the pulses, and pulse wave pattern-related information, as well as other information, discussed below. Specifically, the pulse wave data can be used to calculate the blood flow of the patient and can also be used to calculate coefficients used in the calculation of the blood glucose level of the patient based on comparison to sample waveforms within a library, as discussed below.

A temperature sensor was also applied to calculate the thermal conductivity of the blood. It is of note that in some embodiments, the temperature sensor is part of the pulse oximeter or is attached thereto although this is not necessary in all embodiments.

By making the above three measurements, the patient's blood glucose level can be calculated, as discussed below.

As discussed herein, according to one aspect of the invention, there is provided a non-invasive method of determining blood glucose level in a patient comprising: calculating dissipated heat by measuring room temperature and body temperature of the patient; calculating blood oxygen saturation; determining a first glucose value using the calculated dissipated heat and the blood oxygen saturation; calculating a glucose coefficient using data obtained from a pulse wave taken from the patient using a pulse oximeter; and determining blood glucose level using the first glucose value and the glucose coefficient.

In another aspect of the invention, there is provided a method of determining blood glucose level of an individual comprising: determining body temperature of the individual; determining blood oxygen saturation; calculating blood flow for the individual using a pulse waveform from the individual; calculating a body heat coefficient and a blood flow coefficient by comparing the waveform from the individual to at least one waveform from a library of waveforms; and calculating the blood glucose level of the patient from the body temperature, blood flow, oxygen saturation, body heat coefficient and blood flow coefficient.

The blood sugar concentration or blood glucose level is the amount of glucose present in blood. The mean normal level in humans is about 5.5 mM (5.5 mmol/L or 100 mg/dL). The normal blood glucose level for non-diabetics should be between 70 and 100 mg/dL. The blood glucose target range for diabetics should be 70-130 mg/dL before meals and less than 180 mg/dL after eating. Blood sugar levels that are persistently high are referred to as hyperglycemic and diabetes is characterized by persistent hyperglycemia.

The oxidation of glucose supplies energy to cells and also results in the emission of heat. Therefore, the quantity of dissipated heat can be correlated to the quantity of glucose and oxygen. Based on this, the metabolic heat conformation (MHC) has been developed to monitor blood glucose level. The supplied oxygen can be calculated by the blood oxygen level and blood flow rate. The quantity of dissipated heat can be calculated by:

H=f(G,BF,O)

where H is the quantity of dissipated heat, G is the glucose level, BF is the blood flow rate and O is the degree of blood oxygen saturation. If H, BF and O can be determined, glucose level can also be calculated.

With the skin temperature and room temperature, the transferred heat is

C=h_{c}(T_{S}−T_{A})

Where C is the quantity of heat transferred, h_{c} is the coefficient of heat transferred by Convection, T_{S} is the absolute temperature of the surface and T_{A} is the ambient temperature.

The degree of blood oxygen saturation can be measured by a pulse oximeter. Therefore, the glucose level can be calculated by the heat, blood flow rate and the blood oxygen saturation. In some embodiments, the blood glucose level is calculated using the following formula, although other suitable formulae may be used or may be derived by one of skill in the art:

G=A+B*H+C*BF+D*O

Where:

G is the blood glucose level

A is the thread, which is a constant value incorporated into the equation. In one embodiment shown in the examples, “A” is 0.2.

B is the body heat coefficient, which is determined by comparing the waveform of the individual or patient to a waveform in a library of waveforms that was taken from an individual with the most similar age and body weight as the patient;

H is the body temperature. In the embodiment shown in the examples, H is the body temperature in degrees Celsius minus 30 which provides the “absolute” body temperature for this calculation;

C is the coefficient of blood flow which is also calculated using a waveform from the library as discussed below;

BF is the blood flow which is calculated from the waveform data;

O is the blood oxygen level which is calculated using the pulse oximeter; and

D is the coefficient of blood oxygen which is a constant as shown in the examples.

However, this is the first calculation to obtain G₁.

Furthermore, the pulse wave form extracted using the pulse oximeter is also related to blood glucose level. Accordingly, the second calculation of glucose level coefficient C can be calculated using pulse wave analysis:

Since pulse data is two dimensional time serial data, mining techniques for time serial data analysis can be applied to the pulse data. The waveforms can be categorized based on the similarity between the testing waveform (from the patient) and from a plurality of well classified sample waveforms (for example from a library). As discussed above, these waveforms may be classified according to age and weight of the individuals from which they were taken or by other means. As will be apparent to one of skill in the art, these library waveforms also include information regarding the blood flow, body heat and blood glucose of the individual from which they were taken (at the time at which the waveform data was taken) as this information is used in the calculation of the blood flow coefficient and the blood oxygen coefficient and in some embodiments, the blood glucose coefficient, as discussed herein. Specifically, as discussed herein, the calculated distance between the patient's stable waveform and the closest age and body weight comparable waveform is used to calculate these coefficients. Because the waveforms have same structure: taller systolic component with lower diastolic component following, the similarity calculation can achieve high accuracy. It can be measured by the total distance of corresponding points between the sample (or library) waveform and the testing (or patient) waveform warping.

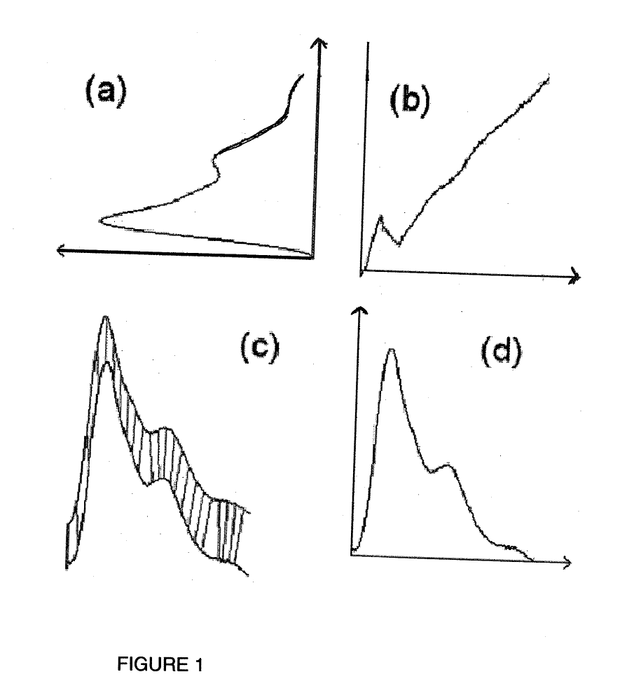

For example, FIG. 1 shows the distance of two waveforms. Panels (a) and (d) are the two waveforms which are being compared. Panel (b) shows point to point difference while panel (c) shows the point based warping result based on dynamic time warping. By determining the distance between the two panels, the coefficient of blood flow can be determined, as discussed herein.

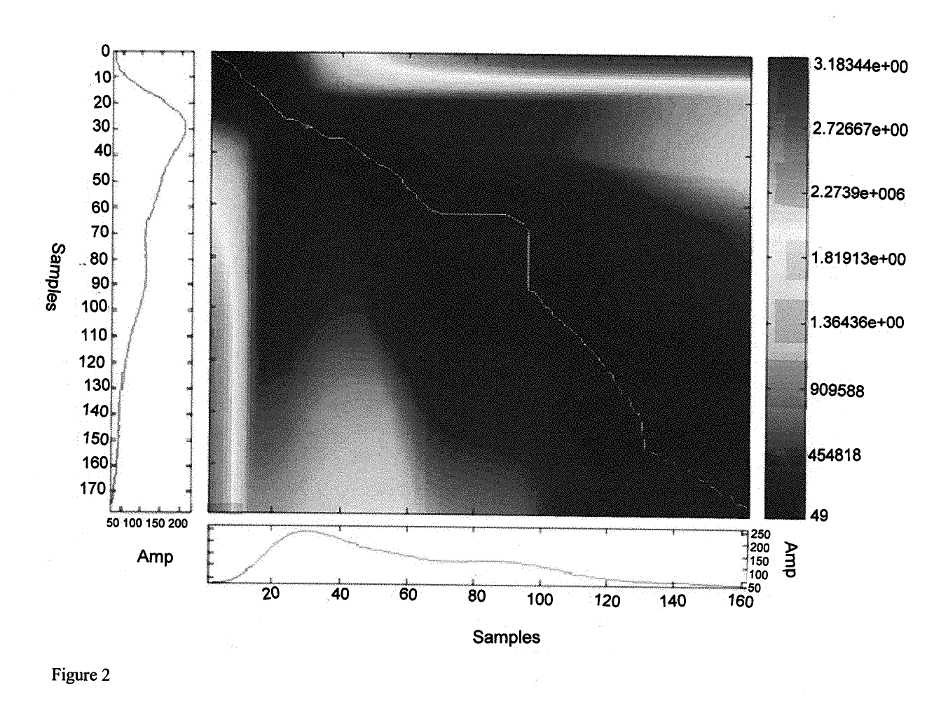

FIG. 2 is a matrix of point to point distance between two pulse wave forms shown in FIG. 1.

A sample waveform is denoted as {xₜ(j),1≤j≤J}, and an unknown frame of the signal as {x(i), 1≤i≤I}. The purpose of the time warping is to provide a mapping between the time indices i and j such that a time registration between the waveforms is obtained. We denote the mapping by a sequence of points c=(i,j), between i and j as

M={c(k), 1≤k≤K}

where c(k)=(i(k),j(k)) and {x(i),1≤i=I} is testing data, {xₜ(j),1≤j≤J} is the template data.

Warping function finds the minimal distance between two sets of data:

d(c(k))=d(i(k), j(k))=∥x(i(k))−xₜ(j(k))∥₂

The smaller the value of d, the higher the similarity between x(i) and xₜ(j)

The optimal path minimize the accumulated distance D_{T}:

$${D}_{T} = \begin{matrix}min \\ \{ M \}\end{matrix} {\sum }_{k = 1}^{K} d ( c ( k ) ) w ( k )$$

where w(k) is a non-negative weighting coefficient.

To find the optimal path, the following calculation needs to be performed:

D(c(k))=d(c(k))+min(D(c(k−1)))

where D(c(k)) represents the minimal accumulated distance

There are two restrictions for warping pulse wave:
1. Monotonic Condition: i(k−1)≤i(k) and j(k−1)≤j(k)
2. Continuity condition: i(k)| i(k−1)≤1 and j(k)| j(k−1)≤1

The symmetric DW equation with slope of 1 is:

$$D ( c ( k ) ) = d ( c ( k ) ) + min ( \begin{matrix}D ( i ( k - 1 ) , j ( k - 2 ) ) + 2 d ( i ( k ) , j ( k - 1 ) ) \\ D ( i ( k - 1 ) , j ( k - 1 ) ) + 2 d ( c ( k ) ) \\ D ( i ( k - 2 ) , j ( k - 1 ) ) + 2 d ( i ( k - 1 ) , j ( k ) )\end{matrix} )$$

The optimal accumulated distance is normalized by (i+j) for symmetric form.

Based on each pulse wave category by distance measurement, a related glucose coefficient C_{C} will be determined.

Then, the adjusted glucose level is determined by:

G=C_{C}*G₁

According to an aspect of the invention, there is provided a non-invasive method for determining blood glucose level in a patient comprising:
providing a system comprising a temperature sensor and a pulse oximeter comprising a pulse wave sensor;
connecting the pulse oximeter and the temperature sensor to a patient who suffers from or is at risk of developing type I or type II diabetes;
collecting pulse wave data from the patient using the pulse wave sensor;
determining blood flow of the patient from the pulse wave data;
measuring blood oxygen saturation using the pulse oximeter;
using the temperature sensor to calculate dissipated heat from the patient;
calculating a first glucose value from the dissipated heat and the blood oxygen saturation;
calculating a glucose coefficient from the pulse wave data; and
calculating the blood glucose level by multiplying the first glucose value and the glucose coefficient; and
reporting the blood glucose level of the patient to the patient for maintaining glycaemia metabolic control,
said patient taking insulin if hyperglycemic or taking a small amount of carbohydrate if hypoglycemic.

As will be appreciated by one of skill in the art, the above-described method can also be considered to be a method for managing blood glucose in an individual in need of such treatment, for example, an individual who has type I or type II diabetes or who is at risk of developing type I or type II diabetes.

The pulse wave data may be analyzed to extract the size of each pulse, the distance between the pulses and pulse wave pattern-related data.

In some embodiments, only the temperature sensor is used to calculate dissipated heat.

In some embodiments, the pulse wave sensor is placed on a finger of the patient.

The blood flow may be determined by analyzing the pulse wave shape.

The temperature sensor may be part of the pulse oximeter.

In some embodiments, the temperature sensor is arranged to measure skin temperature of the patient and room temperature.

Preferably, the pulse wave data comprises stable waveforms. As discussed above, the waveform is considered to be stable when at least 10 consecutive waveforms or contours vary in height, shape and/or size by less than 5%.

In some embodiments, when the pulse waveforms are stable, the pulse wave sensor stops receiving signals and analyzes the data.

According to another aspect of the invention, there is provided a non-invasive method for determining blood glucose level in a patient comprising:
providing a system comprising a library of waveforms; a temperature sensor; and a pulse oximeter comprising a pulse wave sensor;
connecting the pulse oximeter and the temperature sensor to a patient who suffers from or is at risk of developing type I or type II diabetes;
collecting pulse wave data from the patient using the pulse wave sensor;
determining blood flow of the patient from the pulse wave data;
determining a blood flow coefficient for the patient by waveform warping of the pulse wave data with a waveform from the library of waveforms corresponding to an individual of similar age and body weight as the patient;
measuring blood oxygen saturation using the pulse oximeter;
using only the temperature sensor to calculate dissipated heat from the patient; determining a body heat coefficient by comparison of the pulse wave data with the waveform from the library;
calculating blood glucose level for the patient using the dissipated heat, the body heat coefficient, the blood flow, the blood flow coefficient and the oxygen saturation; and
reporting the blood glucose level of the patient to the patient for maintaining glycaemia metabolic control,
said patient taking insulin if hyperglycemic or taking a small amount of carbohydrate if hypoglycemic.

The pulse wave data may be analyzed to extract the size of each pulse, the distance between the pulses and pulse wave pattern-related data.

In some embodiments, the method includes calculating blood glucose level for the patient by multiplying the dissipated heat and the body heat coefficient; multiplying the blood flow by the blood flow coefficient; and adding these values and the oxygen saturation together.

The pulse wave sensor may be placed on a finger of the patient.

The blood flow may be determined by analyzing a pulse wave shape.

The temperature sensor may be part of the pulse oximeter.

The temperature sensor may be arranged to measure skin temperature of the patient and room temperature.

The pulse wave data may comprise stable waveforms. Preferably, the pulse wave data comprises stable waveforms. As discussed above, the waveform is considered to be stable when at least 10 consecutive waveforms or contours vary in height, shape and/or size by less than 5%.

In some embodiments, when the pulse waveforms are stable, the pulse wave sensor stops receiving signals and analyzes the data.

In some embodiments, the blood flow coefficient is calculated by measuring total distance between the patient waveform and the selected library waveform.

Method of Use

As will be appreciated by one of skill in the art, any individual suffering from type 1 or type II diabetes or at risk of developing diabetes, for example, because of familial history, life style or a genetic predisposition can use this system. As discussed above, blood glucose level should be monitored at least 4 times per day based on clinic guidelines. Significantly and advantageously, the non-invasive blood glucose level monitoring system described herein can be used without causing discomfort to patients.

This blood glucose level monitoring system includes two parts: a temperature sensor and a pulse oximeter. The temperature sensor measures skin temperature and room temperature. The temperature data is sent to the control unit for further processing, as discussed above.

The pulse oximeter transmits infrared light and is placed on the index finger of the right hand. The pulse wave sensor detects the blood flow at the index finger and tracks the strength of the flow as pulse wave data. When recording the pulse wave, patients were comfortably seated with the right hand supported; a pulse wave sensor was applied to the index finger of the right hand. Only the appropriate and stable contour of the pulse wave was recorded.

The data collected is transmitted to a general use computer or to a dedicated control unit for analysis.

In some embodiments, the device has a USB connection to a computer for data collection. In these embodiments, data is transferred with transmit/receive buffers and modem handshake signals at USB 2.0 full speed. The infrared sensor at finger clip can monitor the transmittance of the finger and generate byte values according to that.

In these embodiments, a time serial is collected at the rate of 200 points per second. The calculation for time interval between two points is based on this rate. The program will link all the points as the graph of a pulse wave. Similar waveforms with normal components (e.g. systolic components and diastolic components) indicate that the waveforms are stable and appropriate as discussed above and the device is directed to stop receiving signals and instead to analyze the data. Temperature data is also transmitted to the computer by USB.

As will be appreciated by one of skill in the art, other methods of recording, reporting and analyzing data known in the art may be used.

For example, a smart phone with a Bluetooth protocol may be used to transfer pulse data and temperature data.

In another example, the system includes two modules to handle data acquisition, transfer and local storage. The calculated blood glucose information is then transmitted to a Control Center for further action.

The invention will now be further illustrated by way of examples. However, the invention is not necessarily limited by the examples.

### Example 1

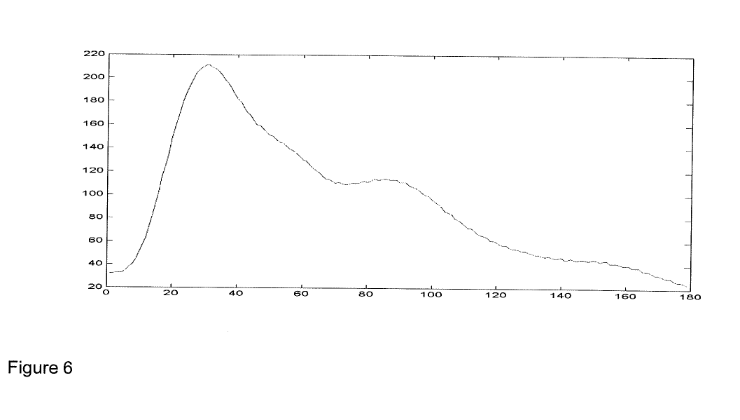

The blood glucose level of a 38 year old patient is calculated by measuring skin temperature (36.2 C) and recording pulse wave data as described above, as shown in FIG. 6.

0.2(thread, calculation adjustment constant)+0.8(body heat coefficient, calculated by waveform comparison)*6.2(absolute skin temperature (36.2−30))+0.11(blood flow coefficient, calculated by waveform comparison)*7.2(blood flow rate, calculated by waveform analysis)+0.02(blood oxygen coefficient, constant)*98 (blood oxygen measured by pulse oximeter)=7.912

Thus, using the above-described method, the calculated blood glucose value is 7.912 mmol/L. For comparison purposes, a prior art invasive method was used and the measured glucose value by that method was determined to be 8.0 mmol/L.

While the preferred embodiments of the invention have been described above, it will be recognized and understood that various modifications may be made therein, and the appended claims are intended to cover all such modifications which may fall within the spirit and scope of the invention.

1. A non-invasive method for determining blood glucose level in a patient comprising:
providing a system comprising a temperature sensor and a pulse oximeter comprising a pulse wave sensor;
connecting the pulse oximeter and the temperature sensor to a patient who suffers from or is at risk of developing type I or type II diabetes;
collecting pulse wave data from the patient using the pulse wave sensor;
determining blood flow of the patient from the pulse wave data;
measuring blood oxygen saturation using the pulse oximeter;
using the temperature sensor to calculate dissipated heat from the patient;
calculating a first glucose value from the dissipated heat and the blood oxygen saturation;
calculating a glucose coefficient from the pulse wave data; and
calculating the blood glucose level by multiplying the first glucose value and the glucose coefficient; and
reporting the blood glucose level of the patient to the patient for maintaining glycaemia metabolic control,
said patient taking insulin if hyperglycemic or taking a small amount of carbohydrate if hypoglycemic.

2. The method according to claim 1 including analyzing the pulse wave data to extract the size of each pulse of the pulse wave data, the distance between each pulse of the pulse wave data and pulse wave pattern-related data.

3. The method according to claim 1 wherein only the temperature sensor is used to calculate dissipated heat.

4. The method according to claim 1 including placing the pulse wave sensor on a finger of the patient.

5. The method according to claim 1 including determining the blood flow by analyzing pulse wave shape of the pulse wave data.

6. The method according to claim 3 wherein the temperature sensor is part of the pulse oximeter.

7. The method according to claim 1 including measuring the skin temperature of the patient and measuring room temperature with the temperature sensor.

8. The method according to claim 2 including analyzing the pulse wave data after stable waveforms have been detected, wherein the waveform is considered to be stable when at least 10 consecutive waveforms or contours vary in height, shape and/or size by less than 5%.

9. The method according to claim 8 wherein when the pulse waveforms are stable, the pulse wave sensor stops receiving signals and analyzes the data.

10. A non-invasive method for determining blood glucose level in a patient comprising:
providing a system comprising a library of waveforms; a temperature sensor;
and a pulse oximeter comprising a pulse wave sensor;
connecting the pulse oximeter and the temperature sensor to a patient who suffers from or is at risk of developing type I or type II diabetes;
collecting pulse wave data from the patient using the pulse wave sensor;
determining blood flow of the patient from the pulse wave data;
determining a blood flow coefficient for the patient by waveform warping of the pulse wave data with a waveform from the library of waveforms taken from an individual of similar age and body weight as the patient;
measuring blood oxygen saturation using the pulse oximeter;
using the temperature sensor to calculate dissipated heat from the patient;
determining a body heat coefficient by comparison of the pulse wave data with the waveform from the library;
calculating blood glucose level for the patient using the dissipated heat, the body heat coefficient, the blood flow, the blood flow coefficient and the oxygen saturation; and
reporting the blood glucose level of the patient to the patient for maintaining glycaemia metabolic control,
said patient taking insulin if hyperglycemic or taking a small amount of carbohydrate if hypoglycemic.

11. The method according to claim 10 including analyzing the pulse wave data to extract the size of each pulse of the pulse wave data, the distance between each pulse of the pulse wave data and pulse wave pattern-related data.

12. The method according to claim 11 including analyzing the pulse wave data after stable waveforms have been detected, wherein the waveform is considered to be stable when at least 10 consecutive waveforms or contours vary in height, shape and/or size by less than 5%.

13. The method according to claim 10 including calculating the blood glucose level for the patient by multiplying the dissipated heat and the body heat coefficient; multiplying the blood flow by the blood flow coefficient; and adding these values and the oxygen saturation together.

14. The method according to claim 10 including placing the pulse wave sensor on a finger of the patient.

15. The method according to claim 10 including determining blood flow by analyzing pulse wave shape of the pulse wave data.

16. The method according to claim 10 wherein only the temperature sensor is used to calculate dissipated heat.

17. The method according to claim 16 wherein the temperature sensor is part of the pulse oximeter.

18. The method according to claim 16 including measuring the skin temperature of the patient and measuring room temperature with the temperature sensor.

19. The method according to claim 10 including calculating the blood flow coefficient by measuring total distance between the patient waveform and the selected library waveform.
